(19) United States
(12) Patent Application Publication
(10) Pub. No.: US 2014/0345623 A1
(43) Pub. Date: Nov. 27, 2014

(54) NASAL DEVICES WITH VARIABLE LEAK PATHS, NASAL DEVICES WITH ALIGNERS, AND NASAL DEVICES WITH FLAP VALVE PROTECTORS

(71) Applicant: VENTUS MEDICAL, INC., San Jose, CA (US)

(72) Inventors: Ryan K. Pierce, San Jose, CA (US); Kenneth Chou, San Jose, CA (US); Matthew Durack, San Jose, CA (US); Mark C. Feldmeier, San Jose, CA (US); Michael L. Favet, San Jose, CA (US); Frank W. Wang, San Jose, CA (US); Arthur Ferdinand, San Jose, CA (US)

(21) Appl. No.: 14/371,392

(22) PCT Filed: Jan. 18, 2013
(86) PCT No.: PCT/US13/22121

§ 371 Date: Jul. 9, 2014

Related U.S. Application Data
(60) Provisional application No. 61/589,071, filed on Jan. 20, 2012.

(51) Int. Cl. A61M 16/20; A61F 5/56

(52) U.S. Cl. CPC A61M 16/208; A61F 5/56; A61M 2210/0618
USPC 128/207.18

(57) ABSTRACT

Improved passive resistance nasal devices for treating a patient (and particularly, but not exclusively, a sleeping patient) that inhibit exhalation more than inhalation. For example, described herein are passive-resistance nasal devices having a variable sized opening leak path that change the size of the leak path opening depending on the pressure extended across the nasal device. Also described herein are passive nasal devices including a deployable insertion guide member. Also described herein are passive nasal devices including an extension member to hold the airflow resistor portion of the nasal device slightly apart from the subject's nose, even as the nasal device itself may be secured against the nose or nostril openings. Methods of operating these nasal devices and methods of treating patients using these devices are also described.

### CROSS REFERENCE TO RELATED APPLICATIONS

This patent application may be related to each of the following patents or patent applications, each of which is herein incorporated by reference in its entirety: U.S. Pat. No. 7,856,979 issued on Dec. 28, 2010 and titled: NASAL RESPIRATORY DEVICES; U.S. Pat. No. 7,798,148 issued on Sep. 21, 2010 and titled: RESPIRATORY DEVICES; U.S. Pat. No. 7,735,491 issued on Jun. 15, 2010 and titled: METHODS OF TREATING RESPIRATORY DISORDERS; U.S. Pat. No. 7,735,492 issued on Jun. 15, 2010 and titled: NASAL RESPIRATORY DEVICES; U.S. Pat. No. 7,992,564 issued on Aug. 9, 2011 and titled: RESPIRATORY DEVICES; U.S. Pat. No. 7,806,120 issued on Oct. 5, 2010 and titled: NASAL RESPIRATORY DEVICES FOR POSITIVE END-EXPIRATORY PRESSURE; U.S. Pat. No. 6,722,360 issued on Apr. 20, 2004 and titled: METHODS AND DEVICES FOR IMPROVING BREATHING IN PATIENTS WITH PULMONARY DISEASE; U.S. Pat. No. 7,334,581 issued on Feb. 26, 2008 and titled: METHODS AND DEVICES FOR IMPROVING BREATHING IN PATIENTS WITH PULMONARY DISEASE; U.S. Pat. No. 7,992,563 issued on Aug. 9, 2011 and titled: METHODS AND DEVICES FOR IMPROVING BREATHING IN PATIENTS WITH PULMONARY DISEASE; U.S. Pat. No. 7,506,649 issued on Mar. 24, 2009 and titled: NASAL DEVICES; U.S. Pat. No. 7,987,852 issued on Aug. 2, 2011 and titled: NASAL DEVICES; U.S. Pat. No. 8,020,700 issued on Sep. 20, 2011 and titled: PACKAGING AND DISPENSING NASAL DEVICES; U.S. patent application Ser. No. 12/955,633 filed on Nov. 29, 2010 and titled: NASAL RESPIRATORY DEVICES; U.S. patent application Ser. No. 11/759,916 filed on Jun. 7, 2007 and titled: LAYERED NASAL DEVICES; U.S. patent application Ser. No. 12/877,836 filed on Sep. 8, 2010 and titled: NASAL RESPIRATORY DEVICES FOR POSITIVE END-EXPIRATORY PRESSURE; U.S. patent application Ser. No. 12/884,140 filed on Sep. 16, 2010 and titled: SEALING NASAL DEVICES FOR USE WHILE SLEEPING; U.S. patent application Ser. No. 12/884,146 filed on Sep. 16, 2010 and titled: NASAL DEVICES FOR USE WHILE SLEEPING; U.S. patent application Ser. No. 12/884,151 filed on Sep. 16, 2010 and titled: NASAL DEVICES WITH RESPIRATORY GAS SOURCE; U.S. patent application Ser. No. 12/885,359 filed on Sep. 17, 2010 and titled: METHODS OF TREATING A SLEEPING SUBJECT; U.S. patent application Ser. No. 12/885,366 filed on Sep. 17, 2010 and titled: METHODS OF TREATING A DISORDER BY INHIBITING EXPIRATION; U.S. patent application Ser. No. 12/885,370 filed on Sep. 17, 2010 and titled: QUIET NASAL RESPIRATORY DEVICES; U.S. patent application Ser. No. 12/141,875 filed on Jun. 18, 2008 and titled: ADHESIVE NASAL RESPIRATORY DEVICES; U.S. patent application Ser. No. 13/164,705 filed on Jun. 20, 2011 and titled: METHODS AND DEVICES FOR IMPROVING BREATHING IN PATIENTS WITH PULMONARY DISEASE; U.S. patent application Ser. No. 11/941,915 filed on Nov. 16, 2007 and titled: ADJUSTABLE NASAL DEVICES; U.S. patent application Ser. No. 11/941,913 filed on Nov. 16, 2007 and titled: NASAL DEVICE APPLICATORS; U.S. patent application Ser. No. 12/044,868 filed on Mar. 7, 2008 and titled: RESPIRATORY SENSOR ADAPTERS FOR NASAL DEVICES; U.S. patent application Ser. No. 13/164,684 filed on Jun. 20, 2011 and titled: NASAL DEVICES; U.S. patent application Ser. No. 12/364,264 filed on Feb. 2, 2009 and titled: CPAP INTERFACE AND BACKUP DEVICES; U.S. patent application Ser. No. 13/212,948 filed on Aug. 18, 2011 and titled: PACKAGING AND DISPENSING NASAL DEVICES; U.S. patent application Ser. No. 12/329,895 filed on Dec. 8, 2008 and titled: DELAYED RESISTANCE NASAL DEVICES AND METHODS OF USE; U.S. patent application Ser. No. 12/405,837 filed on Mar. 17, 2009 and titled: NASAL DEVICES WITH NOISE-REDUCTION AND METHODS OF USE; U.S. patent application Ser. No. 12/485,750 filed on Jun. 16, 2009 and titled: ADJUSTABLE RESISTANCE NASAL DEVICES; U.S. patent application Ser. No. 13/062,888 filed on May 17, 2011 and titled: NASAL DEVICES, SYSTEMS AND METHODS; U.S. patent application Ser. No. 12/941,734 filed on Nov. 8, 2010 and titled: NASAL DEVICES HAVING A SAFE FAILURE MODE AND REMOTELY ACTIVATABLE; U.S. patent application Ser. No. 13/035,524 filed on Feb. 25, 2011 and titled: NASAL DEVICES INCLUDING LAYERED NASAL DEVICES AND DELAYED RESISTANCE ADAPTERS FOR USE WITH NASAL DEVICES; and U.S. patent application Ser. No. 13/117,933 filed on May. 27, 2011 and titled: LAYERED NASAL RESPIRATORY DEVICES.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This provisional patent application describes nasal respiratory devices. These nasal respiratory devices typically include a passive airflow resistor configured to inhibit exhalation more than inhalation, and may be configured to include one or more variable leak pathways that are open even when the airflow resistor is otherwise closed. Also described are nasal respiratory devices with an alignment mechanism. Also described are nasal respiratory devices that are configured to protect the airflow resistor.

### BACKGROUND

A nasal respiratory device as described herein may be used to treat a subject with a respiratory disorder, including, but not limited to, sleeping disorders such as apnea (including obstructive sleep apnea (OSA), central sleep apnea, Cheyne-Stokes breathing), COPD, snoring, gastroesophageal reflux disease and the like.

Nasal respiratory devices have been well-described in the following patents and patent applications, each of which was previously incorporated in its entirety: U.S. patent application Ser. No. 11/298,339, titled, “RESPIRATORY DEVICES,” and filed on Dec. 8, 2005; U.S. patent application Ser. No. 11/805,496, titled, “NASAL RESPIRATORY DEVICES,” and filed on May 22, 2007; U.S. patent application Ser. No. 11/759,916, titled, “LAYERED NASAL DEVICES,” and filed on Jun. 7, 2007; U.S. patent application Ser. No. 12/141,875, titled, “ADHESIVE NASAL RESPIRATORY DEVICES,” and filed on Jun. 18, 2008; U.S. patent application Ser. No. 11/811,401, titled, “NASAL RESPIRATORY DEVICES FOR POSITIVE END-EXPIRATORY PRESSURE,” and filed on Jun. 7, 2007; U.S. patent application Ser. No. 11/941,915, titled, “ADJUSTABLE NASAL DEVICES,” and filed on Nov. 16, 2007; U.S. patent application Ser. No. 11/941,913, titled, “NASAL DEVICE APPLICATORS,” and filed on Nov. 16, 2007; U.S. patent application Ser. No. 11/811,339, titled, “NASAL DEVICES,” and filed on Jun. 7, 2007; U.S. patent application Ser. No. 12/044,868, titled, “RESPIRATORY SENSOR ADAPTERS FOR NASAL DEVICES,” and filed on Mar. 7, 2008; U.S. patent application Ser. No. 12/369,681, titled, “NASAL DEVICES,” and filed on Feb. 11, 2009; U.S. patent application Ser. No. 12/364,264, titled, “CPAP INTERFACE AND BACKUP DEVICES,” and filed on Feb. 2, 2009; U.S. patent application Ser. No. 12/329,271, titled, “PACKAGING AND DISPENSING NASAL DEVICES,” and filed on Dec. 5, 2008; U.S. patent application Ser. No. 12/329,895, titled, “DELAYED RESISTANCE NASAL DEVICES AND METHODS OF USE,” and filed on Dec. 8, 2008; U.S. patent application Ser. No. 12/405,837, titled, “NASAL DEVICES WITH NOISE-REDUCTION AND METHODS OF USE,” and filed on Mar. 17, 2009; and U.S. patent application Ser. No. 12/485,750, titled, “ADJUSTABLE RESISTANCE NASAL DEVICES,” and filed on Jun. 16, 2009.

In general, these nasal respiratory devices are configured to inhibit exhalation more than inhalation in a sleeping patient. The resistance to exhalation may be considered “passive,” since it is applied by a passive airflow resistor, rather than relying on the active application of force (e.g., blowing air). The nasal respiratory devices may be configured to provide resistance to either or both exhalation and inhalation within a specified therapeutic range or ranges for the treatment of apnea, snoring, or other disorders. As used herein, a patient may be any subject, human or non-human, in need of the nasal respiratory (“nasal”) devices described herein or in the incorporated references. These devices may be provided as prescription or non-prescription (“over the counter”) devices.

These patents and patent applications generally describe nasal respiratory devices and methods for treating a variety of medical conditions through the use of such devices. These medical conditions include but are not limited to snoring, sleep apnea (obstructive, central, complex and mixed), Cheyne-Stokes breathing, UARS, COPD, hypertension, asthma, GERD, heart failure, and other respiratory and sleep conditions. Such nasal respiratory devices typically induce positive end-expiratory pressure (“PEEP”) and/or expiratory positive airway pressure (“EPAP”), and are adapted to be removably secured in communication with a nasal cavity. Similarly, the respiratory devices described herein may include devices having one or more expiratory resistor valves.

These devices may include an opening (which may form a passageway), an airflow resistor (e.g., valve) in communication with the opening, and a holdfast to secure the device in communication with a nostril, nasal opening and/or nasal passage. For example, the holdfast may be configured to removably secure the respiratory device within (or over or around) the nasal cavity. The airflow resistor (which may be a valve) is typically configured to provide greater resistance during exhalation than during inhalation.

Although these devices have been generally described both functionally and by example, some specific variations of nasal respiratory devices have not previously been described. Thus, it may be beneficial to improve upon the devices, kits and methods previously described, and particularly to more fully develop certain embodiments of nasal devices and methods of arranging, using, manufacturing, inserting and removing nasal respiratory devices. Described below are specific variations of nasal devices, methods of using nasal devices and kits including such nasal devices.

For example, it would be beneficial to provide nasal devices that are adapted for ease of use and/or comfort and/or improved efficacy. For example, it may be beneficial to provide passive nasal devices that have a variable leak pathway to provide a greater inhibition of or resistance to expiratory flow at lower pressures and lower airflow (during later portions of an expiratory cycle) than at higher pressures and higher airflow (e.g., during earlier portions of an expiratory cycle), which may enhance comfort and/or efficacy. It may also be beneficial to provide a passive nasal device that has a reduced resistance to exhalation at the beginning of exhalation but an increased resistance at the end of exhalation, which my help achieve higher residual airway pressure.

In addition, it may be desirable to provide passive nasal respiratory devices that have protection for the airflow resistor or a moving portion of the airflow resistor. For example, in devices in which a flap valve is included as part of the airflow resistor, the nasal device may be configured with a flap valve protector to prevent the flap valve from contacting the subject's nose, nostril, or associated structures (including nostril hairs, and the like). Such devices may be more reliable in their operation.

It may also be helpful to provide devices that are easier to apply to the nose because they include an integral aligner for aligning the airflow resistor with the subject's nostril(s). It may also be desirable to provide nasal devices for which alignment is not necessary.

The nasal devices, kit, systems and methods described herein address many of the potential benefits described above. In general, the nasal devices described herein may be passive nasal devices having a low profile that may be fabricated economically, and may have enhanced comfort and/or ease of use and/or efficacy, while still inhibiting exhalation more than inhalation with a therapeutically relevant range of resistances.

### SUMMARY OF THE DISCLOSURE

The present invention relates to improvements in passive nasal devices.

For example, described herein are passive-resistance nasal devices having a variable opening leak path that changes the size of the leak path opening depending on the pressure extended across the nasal device. Such devices may be more comfortably tolerated by a patient wearing a device. In some variations the nasal devices for applying passive resistance to a patient include: an airflow resistor configured to provide a resistance to exhalation that is greater than the resistance to inhalation; and a variable opening leak path through the device configured so that the size of the leak path opening increases as expiratory pressure increases and decreases as expiratory pressure decreases.

Any of the nasal devices described herein may be configured to have a resistance to exhalation that is between about 0.002 and about 0.25 cm H₂O/(ml/sec) when measured at 100 mL/sec. Any of the devices described herein may be an adhesive nasal device (e.g., having an adhesive holdfast).

The variable opening leak path may include a flexible membrane, or be formed by one or more flexible (and/or stretchable, conformable, etc. membranes). For example, the variable opening leak path may comprise a pair of membranes, wherein at least one of the membranes is configured to slide relative to the other as expiratory pressure increases and decreases.

In general, the variable opening leak path responds to the pressure applied across the airflow resistor by changing the gap or leak between through the airflow resistor present during exhalation (e.g., or present during both exhalation and inhalation). In some variations the nasal devices include a leak path comprising a variable opening leak path having a membrane with a spiral of curved or linear cuts. The spiral may be formed by two or more curves (e.g., c-shaped or s-shaped curves) extending around a central region.

Any of the nasal devices described herein may be configured to have an airflow resistor comprising a flap valve having at least one flap. Further, any of the variations described herein may be configured as single-nostril devices (configured to secure the device to a single nostril) or whole-nose devices (configured to secure the device to both nostrils). In some variations, the devices include a holdfast region configured to secure the device in communication with both nostrils (whole nose device) or a single nostril (single nostril device).

Also described herein are passive resistance nasal devices for use while sleeping, the device comprising: an airflow resistor configured to provide a resistance to exhalation that is greater than the resistance to inhalation; and a variable opening leak path comprising a membrane having a plurality of cuts forming leak path openings through the membrane arranged in a spiral pattern and configured so that the size of the leak path openings increase as expiratory pressure increases and decreases as expiratory pressure decreases.

The passive-resistance devices described herein typically modify the respiration through the patient's nose, and particularly a sleeping patient's nose, inhibiting exhalation more than inhalation, without the addition of any pressurized breathing gas. In operation, any of the nasal devices including a variable opening leak path may be used as part of a method of treating a patient, or as part of a method of treating a sleeping patient. For example, described herein are methods of treating a sleeping patient, the method comprising the steps of: applying a passive nasal device in communication with each or both of the patient's nostrils without covering the patient's mouth; inhibiting exhalation through the patient's nose more than inhalation through the nose; and changing the size of a leak path opening through the nasal device during exhalation based on the pressure applied across the nasal device during exhalation.

Also described herein are passive nasal devices for treating a patient (and particularly but not exclusively a sleeping patient) including a deployable insertion guide member. For example, described herein are passive nasal devices comprising: an airflow resistor configured to inhibit exhalation more than inhalation; a holdfast at least partially surrounding the airflow resistor and configured to secure the nasal device to a patient's nose; and an insertion guide member configured to be deployed from a collapsed position adjacent to the holdfast to an expanded position for placement at least partially within the patient's nostril. In some variations, the airflow resistor comprises a flap valve, and the holdfast may be configured as an adhesive holdfast.

In general, the insertion guide member may be configured to change from a flat (e.g., in-line with the plane of the holdfast and/or airflow resistor element) to an extended configuration that can be inserted into the nose to guide the placement of the airflow resistor relative to the nostril opening and/or protect the airflow resistor. For example, in some variations, the insertion guide comprises a hinged member, e.g., the insertion guide may comprise a pair of hinged arches. The insertion guide member may include a pair of curving members. Thus, the insertion guide may be configured to be deployed from a plane parallel to the airflow resistor to a plane at an angle with the airflow resistor

Any of the devices described herein may be formed, packaged, or held on a support backing or support card to hold the device. The support card may include an indicator, indicating how to deploy the insertion guide member.

Also described herein are methods of applying a nasal device for use while sleeping, wherein the nasal device comprises an airflow resistor configured to inhibit exhalation more than inhalation and a holdfast configured to secure the nasal device to the patient's nose, the method comprising: deploying an insertion guide member from a collapsed position adjacent to the holdfast to an expanded position extending from the device; placing the insertion guide at least partially within the patient's nostril; and securing the nasal device to the patient's nose using the holdfast.

Also described herein are passive nasal devices including an extension member to hold the airflow resistor portion of the nasal device slightly apart from the subject's nose, even as the nasal device itself may be held snugly against the nose. For example, described herein are passive nasal devices, the nasal devices comprising: an airflow resistor configured to inhibit exhalation more than inhalation; a holdfast configured to secure the nasal device to a patient's nose; and an extension member between the holdfast and the airflow resistor configured to position the airflow resistor between about 1.5 mm and about 25 mm from the nose.

The extension member may be a ring of material coupled to the holdfast at a first end, wherein the extension member surrounds the airflow resistor at a second end of the extension member. In some variations, the extension member forms a passageway between the holdfast and the airflow resistor.

The holdfast may be an adhesive holdfast, and may be configured as a whole-nose or single-nostril holdfast, to secure the nasal device to both of a patient's nostrils or a single nostril, respectively.

In general, the extension member may comprise a ring having a central opening, or an oval-shaped ring having a central opening (though any shape with an opening through which air may pass may be used). For example, the extension member may have a central opening with a diameter that is greater than about 8.5 mm.

The extension member may be formed of any appropriate material. For example, the extension member may be formed of (or at least partially formed of) a foam material. In some variations, the extension member is formed of a rigid material. The extension member may be compliant or rigid. In some variations, the extension member comprises a leak path.

Also described herein are passive nasal devices (which may be configured for use while sleeping) that include: an airflow resistor comprising a flap valve configured to inhibit exhalation more than inhalation; an adhesive holdfast configured to secure the nasal device to a patient's nose; and an extension member having an opening, wherein a first end of the extension member is connected to the holdfast, and the airflow resistor extends across the opening at a second end of the extension member separated from the first end by more than about 1.5 mm.

Any of these devices may be used as part of a method of treating a patient, including sleeping patients. For example, described herein are methods of treating a sleeping patient, the method comprising: adhesively securing a first end of a passive nasal device in communication with each or both of the patient's nostrils without covering the patient's mouth, wherein the passive nasal device comprises a flap valve and an extension member configured to position the flap valve between about 1.5 and about 25 mm from the patient's nostril opening; and inhibiting exhalation through the patient's nose more than inhalation through the nose.

### BRIEF DESCRIPTION OF THE DRAWINGS

FIGS. 1A to 1C illustrate one variation of a variable opening leak path.

FIGS. 2A-2D one variation of passive nasal device having a variable opening leak path.

FIGS. 3A-3D show another variation of a nasal device having a variable opening leak path.

FIG. 4 illustrates operation of another variation of a variable opening leak path configured as a spiral.

FIGS. 5A and 5B illustrate one variation of a passive nasal device having the variable opening leak path shown in FIG. 4.

FIG. 6 is a graph comparing the pressure and flow relationships of a variable opening leak path device (“array resistor”) to a fixed sized opening leak path (“hole resistor”) when both devices are set to allow flow of ˜100 mL/s at a pressure of ˜0.5 cm H₂O.

FIG. 7 is similar to FIG. 6, but shows the pressure and flow relationships of a variable opening leak path device (“array resistor”) to a fixed sized opening leak path (“hole resistor”) when both devices are set to allow flow of ˜100 mL/s at a pressure of ˜1 cm H₂O.

FIG. 8 is similar to FIG. 6, but shows the pressure and flow relationships of a variable opening leak path device (“array resistor”) to a fixed sized opening leak path (“hole resistor”) when both devices are set to allow flow of ˜100 mL/s at a pressure of ˜2-3 cm H₂O.

FIG. 9 is similar to FIG. 6, but shows the pressure and flow relationships of a variable opening leak path device (“array resistor”) to a fixed sized opening leak path (“hole resistor”) when both devices are set to allow flow of 100 mL/s at a pressure of ˜6-8 cm H₂O.

FIG. 10 shows one variation of a nasal device including a deployable insertion guide attached to a support backing.

FIG. 11 shows the nasal device of FIG. 10, without the deployment guides indicated.

FIG. 12A illustrates one variation of a deployable insertion guide configured as a deformable plastic cut-out region.

FIG. 12B illustrates deployment of the deployable insertion guide shown in FIG. 12A, and FIG. 12C illustrates a nasal device including the deployable insertion guide, showing the insertion guide deployed.

FIG. 13 illustrates one method of deploying insertion guides for two nasal devices prior to applying them to a patient's nose.

FIGS. 14A and 14B show an alignment cone and a deployable insertion guide, respectively.

FIGS. 15A and 15B show another variation of a deployable insertion guide, in an undeployed and deployed state, respectively.

FIG. 15C shows a comparison between nasal devices having a deployed insertion guide (on the right) and an alignment cone (on the left) with the insertion guide in the deployed configuration.

FIGS. 15D and 15E show top and bottom views, respectively of the nasal devices shown in FIG. 15C.

FIGS. 16A-16D illustrate one variation of a nasal device having an extension member to offset the airflow resistor of the nasal device from the adhesive holdfast. FIG. 16A shows a bottom (patient-facing) perspective view, FIG. 16B shows a top perspective view, FIG. 16C shows a bottom view, and FIG. 16D shows a side perspective view, respectively.

### DETAILED DESCRIPTION

Described herein are improved nasal devices. All of the nasal devices illustrated below typically include an airflow resistor configured to passively inhibit exhalation through the device more than inhalation through the device, and a holdfast configured to hold the device securely to the subject's nostril(s) and thereby inhibit exhalation more than inhalation. In some variations the airflow resistor includes one or more flap valves and a flap valve limiter layer. The flap valve limiter layer may prevent the flap valve from substantially opening during exhalation, but allows the flap vale to open relatively freely during inhalation. In many variations the resistance to exhalation is much greater than the resistance to inhalation.

Many of the issued patents and pending patent applications incorporated by reference above describe passive nasal devices; in many of these variations the nasal devices include one or more leak pathways through which air may pass during exhalation, even when the airflow resistor is closed. These leak pathways may include a fixed diameter or sized hole or opening though the nasal device. The openings may pass through the valve(s) of the airflow resistor, the body of the device, some combination of the body and the valve(s), or elsewhere on the device. The devices may be configured so that resistance to airflow through the device is generally greater during exhalation than during inhalation and the resistance to exhalation and the resistance to inhalation during operation remains within therapeutic ranges, as discussed below.

Part I of this disclosure describes variations of nasal devices having a variable leak pathway configured as a flow regulator that modulates the resistance to exhalation; typically these devices are adapted so that the greater the pressure differential across the device during exhalation, the greater the flow through the leak path. For example, in some variations the leak path may include a regulator that enlarges the leak path as pressure increases. In many variations, even though the leak pathway allows a greater “leak” at higher pressures, the overall resistance to exhalation is still greater than that of inhalation, and remains within the therapeutic range of resistances to exhalation, particularly compared to inhalation. In use, as described below, these devices may open (or further open) during the start of exhalation to allow a larger flow (particularly as compared to a fixed-size leak path opening), but close (or further close) as the pressure during exhalation decreases in the later stages of exhalation, thus decreasing the flow through the leak path. Surprisingly, this configuration, in which the airflow through the device during exhalation is greater at higher pressures while the overall resistance to exhalation provided by the device is greater than the resistance to inhalation, may provide therapeutic effects for treating a patient more comfortably and/or effectively than comparable devices having a fixed leak path.

Part II of this disclosure describes improved nasal respiratory devices in which the airflow resistor (e.g., flap valve in some variations) is protected, and/or a placement guide is included.

Part III describes nasal respiratory devices in which a placement guide is unnecessary. These nasal respiratory devices may be considered alignment insensitive, because the passive airflow resistor is separated from the plane of the nostril openings (away from the patient). Such devices may include a holdfast for securing the device over, around and/or slightly within the nasal openings, and a passive airflow resistor connected to the holdfast by a channel that positions the airflow resistor away from the plane of the nasal openings.

In general, any of the nasal devices described herein may be adhesive nasal devices that are configured to adhesively secure to, around, and/or in the nostrils. As mentioned, these devices typically have a greater resistance to exhalation than to inhalation over at least a portion of the respiratory cycle. This resistance is achieved passively (e.g., by using a mechanical valving means), rather than by the application of additional pressurized gas.

In some variations, the nasal device is configured so that there is only nominal resistance through the nasal device during inhalation (e.g., less than about 0.0005 cm H₂O/(ml/sec) at 100 ml/sec, less than about 0.001 cm H₂O/(ml/sec) at 100 ml/sec, less than about 0.005 cm H₂O/(ml/sec) at 100 ml/sec, less than about 0.004 cm H₂O/(ml/sec) at 100 ml/sec, less than about 0.003 cm H₂O/(ml/sec) at 100 ml/sec, less than about 0.002 cm H₂O/(ml/sec) at 100 ml/sec, etc.), but increased resistance to airflow during exhalation (e.g., greater than about 0.001 cm H₂O/(ml/sec) at 100 ml/sec, greater than about 0.003 cm H₂0 at 100 ml/sec, greater than about 0.005 cm H₂O/(ml/sec) at 100 ml/sec, greater than about 0.01 cm H₂0 at 100 ml/sec, greater than about 0.02 cm H₂O/(ml/sec) at 100 ml/sec, greater than about 0.03 cm H₂0 at 100 ml/sec, greater than about 0.04 cm H₂O/(ml/sec) at 100 ml/sec, greater than about 0.05 cm H₂O/(ml/sec) at 100 ml/L, greater than about 0.06 cm H₂O/(ml/sec) at 100 ml/sec, greater than about 0.07 cm H₂O/(ml/sec) at 100 ml/sec, greater than about 0.08 cm H₂O/(ml/sec) at 100 ml/sec, greater than about 0.09 cm H₂O/(ml/sec) at 100 ml/sec, greater than about 0.1 cm H₂O/(ml/sec) at 100 ml/sec, greater than about 0.12 cm H₂O/(ml/sec) at 100 ml/sec, etc.). In some variations the resistance to exhalation may vary with applied pressure, but the resistance to exhalation in these devices is still greater than the resistance to inhalation.

In some embodiments, the resistance to airflow during exhalation may be between a predetermined range of values (e.g., between about 0.002 and about 0.25 cm H₂0/(ml/sec) measured at a flow rate of 100 ml/sec, or between about 0.005 and 0.15 cm H₂O/(ml/sec) when measured at 100 ml/sec. In some variations of the adhesive devices described herein adapted to be used for snoring, the airflow resistor creates a pressure during exhalation that is between about 0.5 cm of H₂0 and about 10 cm H₂0 measured at flow rates of 100 ml/sec, or between about 2 cm H₂0 and about 8 cm H₂0 measured at flow rates of 100 ml/sec, or between about 3 cm H₂0 and about 8 cm H₂0 measured at flow rates of 100 ml/sec, or about 4 cm H₂0 measured at flow rates of 100 ml/sec. In some variations, the pressure during inhalation may be between about 0.0001 cm H₂O and 3 cm H₂O, measured at 100 ml/sec. As is apparent above, in some variations, the therapeutic range of resistance (and particularly expiratory resistances) may overlap or be identical to the other therapeutic resistance ranges described herein.

The devices described herein may be configured to secure completely over the outside of the nose, or in some variations over the outside and partially within the nose. Some variations of these devices may be configured as whole-nose device, e.g., so that the airflow resistor is in communication with both nostrils and nasal breathing from both nostrils passes through the same airflow resistor or airflow resistor region. In some variations, the nasal devices are single-nostril devices, and two devices may be worn at the same time, one on each nostril. The improvements to nasal devices described herein with respect to parts I-III may be incorporated into any of the nasal devices mentioned in above and incorporated by reference.

### Part I: Variable Leak Path Nasal Devices

A passive nasal respiratory device may include an airflow resistor that inhibits exhalation more than inhalation and may also include one or more leak paths. In some variations the leak path is formed as part of the airflow resistor. The airflow resistor typically includes one or more valves (or valving mechanism) for inhibiting airflow through the nasal device more during exhalation than during inhalation. The airflow resistors described herein typically include flap valves, though it should be understood that any appropriate valve may be used, including ball valves, check valves, membrane valves, etc. (including combinations of these). In some variations the airflow resistor includes more than one valve. For example, more than one flap valve, or a valve having multiple leaflets, may be used. The valve is typically open during inhalation, so that in some variations the majority of airflow through the device occurs thought the open airflow resistor during inhalation. Thus, as mentioned, the resistance through the nasal device to inhalation is typically low (e.g., less than about 0.002 cm H₂O measured at a flow rate of 100 ml/L, less than about 0.005 cm H₂O at 100 ml/L, etc.). During exhalation, this airflow resistor typically closes, and flow through the nasal device passes through a leak path or leak pathways.

A leak path may be open during both exhalation and inhalation (e.g., constantly open). Multiple leak paths may be used. In some variations, the leak path is a dedicated opening of a fixed size during both exhalation and inhalation, although any contribution of flow through the leak path during inhalation is usually negligible. For example, a leak path may include an opening through the nasal device that is a hole (referred to as a “hole resistor”). The diameter of the hole may be constant. By contrast, in some variations the nasal device may include a leak path that has an opening though the device that is variable. In some variations this leak path also acts as a valve that can be opened a variable amount during exhalation, depending on the pressure across the nasal device. The valve or valving mechanism for a leak path may be referred to as a flow regulator. Flow regulators may regulate the flow during exhalation over the range of pressures typical to exhalation, so that as exhalation pressure increases the opening of the flow regulator increases size to allow additional flow through the leak pathway. In some variations the flow regulator of the leak path may be distinguished from the valve of the airflow resistor that opens during inhalation, because the valve of the airflow resistor typically opens fully (or nearly fully) with just nominal pressure during inhalation over the typical range of pressures typically to inhalation.

Multiple leak paths may be used. For example, a combination of fixed-size (e.g., hole resistor) and flow regulator leak pathways may be used. In some variations, multiple flow regulator leak pathways may be used as part of a variable leak path (e.g., variable opening leak path).

In some variations, the airflow resistor is integrated with a leak path. For example, the leak path may present on the valve forming the airflow resistor that opens during inhalation. A leak path (or a portion of the overall leak path) through the airflow resistor may be formed as part of the valve forming the airflow resistor that opens during inhalation. In one variation the valve is a dual valve that opens during inhalation with little resistance (e.g., within the therapeutic ranges providing resistance to inhalation as illustrated above) that also opens to provide a leak path during exhalation to achieve the therapeutic range of expiratory resistances described above and opening more as pressure during exhalation increases.

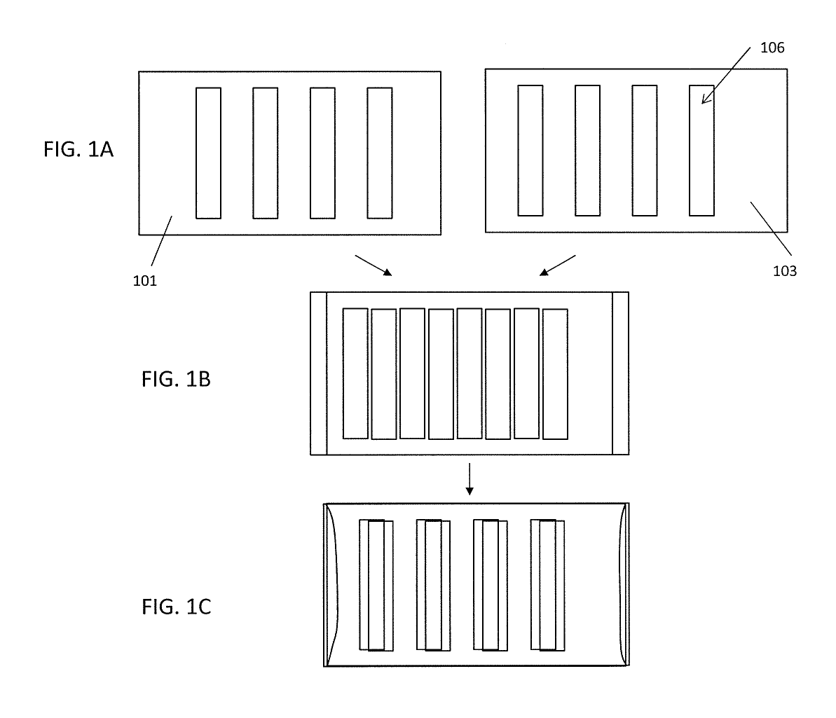

FIGS. 1 to 5B illustrate variations of variable (or variable opening) leak pathways that may be included as part of a passive nasal device to inhibit exhalation more than inhalation. The leak pathways described herein are configured to open more (permitting more flow through the leak path) as the pressure differential produced across the device increases. Any of these devices may be referred to as variable leak path devices (e.g., variable opening leak path devices).

We herein hypothesize that a passive nasal respiratory device may be made more comfortable and/or effective for a user by increasing the flow (and in some variations, reducing the resistance) for airflow through the leak path and thus the device near the beginning of exhalation, when the lungs are inflated, while still maintaining the therapeutic benefits of the device by keeping the overall resistance to exhalation within the therapeutic ranges described herein. For example, at the start of exhalation, the pressure from the lungs is high, particularly compared to the pressure near the end of exhalation. Thus, the resistance to exhalation through the nasal device may be varied depending on the pressure applied to the nasal device as the flow through the leak path is decreased or increased based on the pressure across the device changing the size of the leak path(s).

To achieve this effect, one or more leak path may be configured as a flow regulator that responds to the changing pressure differential across the device during exhalation by increasing flow through the leak pathway at high expiratory pressure and decreasing the flow through the leak pathway at low expiratory pressure may be used. This may be referred to as a variable opening leak path or variable leak path. In some variations this may be achieved by the use of a valve (or flow regulator) through the nasal device forming a leak pathway that increases the opening of the leak pathway as expiratory pressure increases. In some variations the flow regulator includes a deformable or displaceable element(s) that changes configuration in response to pressure across the leak path (e.g., across the device or at least the flow regulator portion of the device). As mentioned above, the variable leak path flow regulators are distinguished from valves that merely open or close in response to a change in pressure across them (which are more typically “on” or “off” above or below a threshold, or have a very narrow, e.g., less than a 0.1 cm H₂O, pressure range between fully open and fully closed states); instead these flow regulators may adjust the opening size over a range of pressures that contains, partly overlaps with, or falls within the pressure range applied across the device during exhalation.

For example, FIGS. 1A-1C illustrates one variation of a flow regulator that may form part of a variable opening leak path as described herein. In this example the leak path includes a two elastic membranes 101, 103 each have multiple small open regions 106 or cut-outs, as shown in FIG. 1A. In this example the cut out regions are rectangular, though a variety of different shapes and configurations may be used. These two (or more) membranes are placed atop one another, and secured in place on two or more (e.g., 3 of the 4) sides. The slots in the neutral position (e.g., without pressure across them) may be overlapping leaving a narrow gap or opening through which air may escape. This is shown in FIG. 1B. When exposed to a pressure differential (e.g., pressurized) such as during exhalation, the membranes may balloon outwards and deform and/or displace relative to each other, resulting in changing the size of the opening between the cut-out regions, as shown in FIG. 1B. In this example, three sides of each membrane may be secured (for the lower membrane, the top, left side, and bottom may be secured; for the upper membrane, the top, right side and bottom may be secured); because opposite sides of the upper and lower membrane are held, the other sides may move relative to each other, leading to the overlapping leak path regions shown in FIG. 1C. As the cut-out regions (slots) align because of the deformation and/or movement of the upper and/or lower membrane, the leak pathway size increases, and the greater the flow through the leak path regulated by this variation of the leak pathway flow regulator. Thus, as the pressure differential increases, the open size of the leak path increases, resulting in a variable leak pathway that acts as a flow regulator.

Other variations of flow regulators for a leak pathway of a nasal device may be used. For example, in some variations, the shape, location and quantity of any cut-out regions (slots) may be varied to achieve a desired relationship between the applied expiratory pressure differential and the cross-sectional area of the available flow path. For example, in some variations the area forming the leak path opening increases as the translation of the flow regulator increases (e.g., the very large cut-out regions shown in FIG. 1A, right and left, overlap more and more as the pressure increases). In some variations the leak path may increase size abruptly, while in other variations the leak path opening may increase in size gradually with pressure increase. Alternatively, in some variations the leak path size may open to a maximum and plateau at higher pressures.

In some variations, the interfacing edges forming the cut-out regions (”slots“) may be configured to prevent catching, snagging or otherwise interfering with the relative movement of the membranes. This may be achieved in some variations by pre-overlapping the membranes. For example, by selectively interfacing angles that are less likely to snag. For example, the edges of the adjacent membranes may be angled relative to each other at a relatively acute angle.

Thus, in general, a flow regulator forming a leak path may be formed of two (or more) membranes that overlap with each other and may be deformed by pressure to provide an increased flow path (leak path) opening size. The site at which the membranes are secured may be varied to control the spatial orientation of the slots when pressurized. In some variations, the shape of the membranes and/or the cut out regions through the membrane may be varied as well. In some variations a single membrane may be used to form a flow regulated leak path, as described in greater detail below; the principle of changing the flow through the leak pathway of the nasal device is similar to that with two membranes as described above.

In some variations, the membrane forming the flow-regulator of the variable leak pathway of a nasal device may be pre-stretched, or pre-compressed so that the membrane(s) form a desired configuration or have a desired behavior when pressurized (during exhalation).

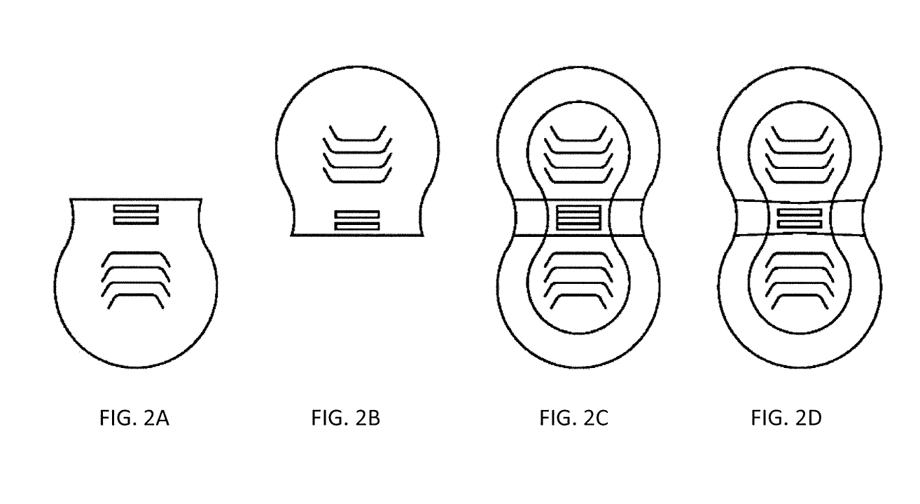

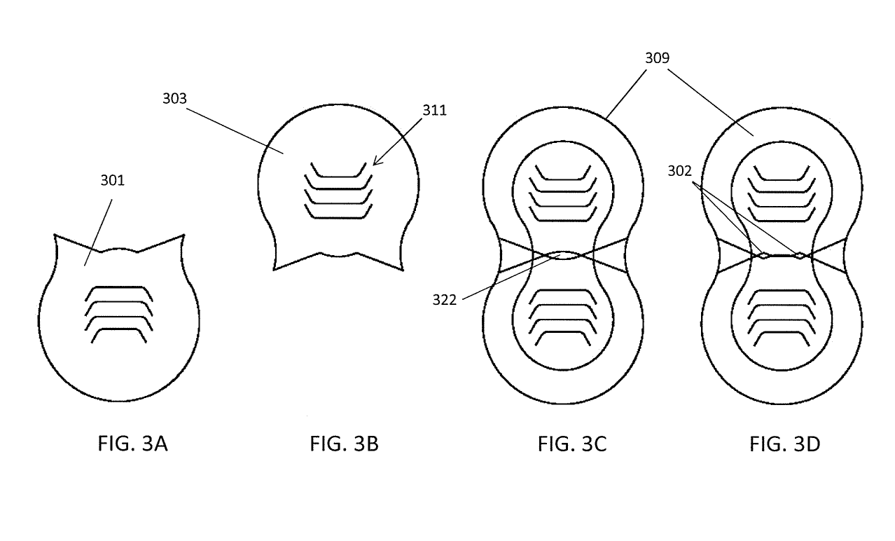

FIG. 2A-2D illustrates the configuration and operation of one variation of an adhesive nasal device configured to include a variable leak pathway as just described. FIG. 2A shows one example of a medial (shown here as the bottom side) region of a nasal device before it is affixed to the lateral (shown in FIG. 2B as the top side) of the nasal respiratory devices, as shown in FIG. 2C. In FIG. 2C the device is shown assembled. This nasal respiratory device includes eight flap valves (arranged above, e.g., medially, and below, e.g. laterally, of midline of the assembled device in FIG. 2C). The region surrounding the perimeter of the device is an adhesive holdfast, which may include a biocompatible adhesive material. Not readily apparent in this example is the valve limiter layer (on the opposite side of this figure) that prevents the flap valves from opening during exhalation. In this example, the central region formed when joining the half of the device shown in FIG. 2A with the half shown in FIG. 2B forms a flow regulator having one or (as in this example) more leak pathways. The leak pathway(s) are formed by the overlap of two or more cut-out regions, similar to that illustrated in FIGS. 1A-1C. In FIG. 2D, the device is illustrated during the start of exhalation, when the cut-out regions are overlapping, thus allowing more flow through the leak path. At the start of inhalation, when the initial pressure is high, the cut-out regions forming the leak path overlap because the ends of the first and second half that overlap may slide against each other until the openings register, as shown in FIG. 3D; as the pressure across the valve during exhalation drops, the membranes slide back, closing (completely or partially) the open region between the membranes.

FIGS. 3A-3D illustrate another variation of a device having a variable opening leak pathway. In this example, there are two parts to the airflow resistor, a first part 301 (in FIG. 3A) and a second part 303 (in FIG. 3B), and the variable-open leak path is formed in the region of overlap between these two membranes, as shown in FIGS. 3C and 3D. In this example, the first airflow resistor element 301of the airflow resistor and the second airflow resistor element of the airflow resistor are configured to at least partially overlap when the first and second airflow resistor elements are assembled into the airflow resistor. Once assembled, an adhesive holdfast 309 is added, as shown in FIG. 3C.

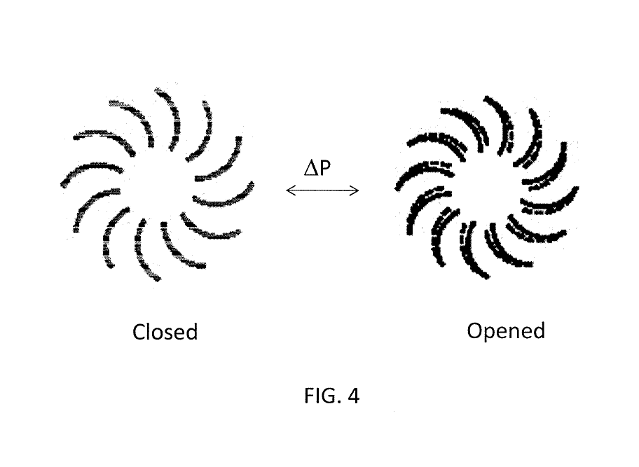

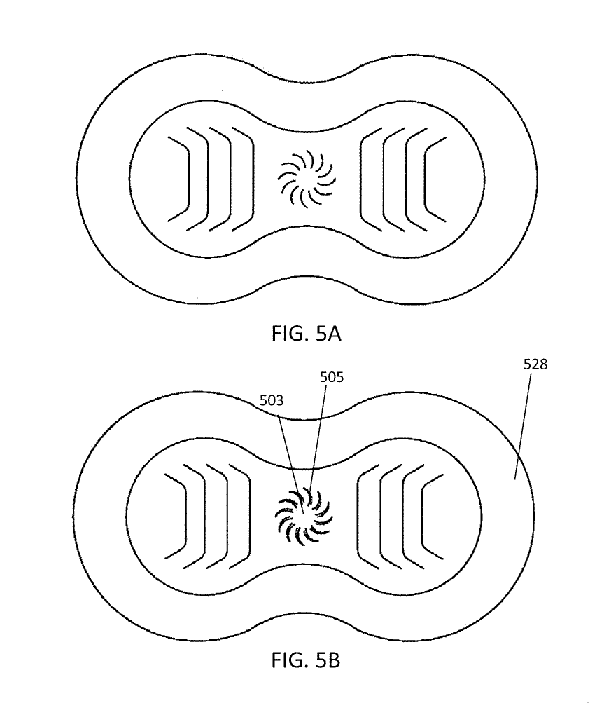

In this variation and some of the variations of variable opening leak paths given herein, over half of the perimeter of the membrane(s) forming the variable opening leak path are secured, while a portion of the edge of the membrane(s) is not secured. In FIGS. 3A-3D much of the perimeter of the two membranes 301, 303 are held in place by the adhesive holdfast and can be secured against the nose. The unsecured portion of one or both of the overlapping unsecured membrane regions forming the leak path may therefore be permitted limited movement relative to the other membrane. In variations of variable opening leak paths having only one membrane, as described in greater detail for FIGS. 4-5B, the entire perimeter of the membrane may be secured; in variations having multiple membranes that move against each other, a region of the membrane may be unsecured, as just described. The unsecured region may be a region of overlap between the two (or more) membranes forming the variable opening leak path. In some variations, less than 50% of the perimeter of the membranes forming the variable opening leak path are secured.

As mentioned above, in some variations of the airflow resistors described herein, and particularly those comprising a flap valve, a flap valve limiter layer may be attached adjacent (e.g., behind) at least the flap valves of the airflow resistors to prevent the flap valve from opening during exhalation. The flap valve limiter layer may be a mesh, or other support structure that prevents the flap valve(s) of the airflow resistor from opening during exhalation by supporting the backs of the flap valves against the pressure of exhalation. In some variations of the devices described herein having variable open leak paths, if a flap valve limiting layer is included it may be not be positioned adjacent to the region forming the variable opening leak path. For example, a flap valve limiting layer may be cut out around this region forming the variable opening leak path (e.g., including an opening, gap, etc.), so that the flap valve limiting layer does not prevent the variable opening leak pathway from opening or closing more or less during exhalation. A flap valve limiter may be, for example, a mesh, support, grid, beam, etc. In FIGS. 3A-3D, the flap valve limiting layer is not shown, but is present behind the flap valves. The flap valve limiting layer does not prevent the first 301 and second 303 airflow resistor membranes from moving relative to each other to open/close during exhalation increasing or decreasing the opening of the leak path as expiratory pressure increases or decreases, respectively.

In operation, the variable opening leak path shown in FIGS. 3A-3D forms a variable opening leak pathway (or flow regulator region) between the first 301 and second 303 airflow resistor elements (membranes) 301, 303. During exhalation, the first 301 and second 300 membranes overlap in the middle region 322 of the nasal device, as shown in FIG. 3C. In this example, the first and second elements are shown as transparent, so that the outer edges of both elements can be seen). In FIG. 3C, the central region overlap 322, and a small amount of air may “leak” in the space between the two membranes. Thus, at rest (or very low expiratory pressures, the leak path is either closed or very small (e.g., the gap between the lateral faces of the first 301 and second 303 elements. The first and second elements 301, 303 forming the airflow resistor in this example are membranes with four flap valves 311 cut into each. During inhalation, the flap valves 311 open. Although air may be inhaled through the leak path as well as these open flap valves, the low inspiratory resistance pathway through the open valves means that the majority of air will be inhaled through these open flaps. However, if the inspiratory pressure increases, the variable opening leak path in this example may also open further, allowing additional airflow through the leak path. FIG. 3C shows the device in the neutral or high expiratory resistance position, with overlap between the first and second elements. During the start of exhalation, as shown in FIG. 3D, when the device is subject to increased expiratory pressure, the flap valves are held shut against the flap valve limiter (not shown), and flow passes primarily through the variable opening leak path(s) 302 formed between the two membranes 301, 303. At higher expiratory pressures, the first and second membranes 301, 303 slide relative to each other and enlarge the leak path opening between the two membranes; the size of the opening vary with pressure. In some variations the size increases as pressure increases and decreases as pressure decreases. However, in some variations the sizes may vary as pressure continues to increase (e.g., increasing over a range of increasing pressures, then decreasing, then increasing again as pressure increases). For example, if the edge of one or both membranes has curved, notches, recessed or cut-out regions so that as the membranes continue to move against each other with increasing (or decreasing) pressure, the size of the opening formed between the two membranes may increase and/or decrease. As example of this was shown in FIGS. 2A-2D, in which increased pressure up to a point causes registration of the cut-out regions between the two membranes; increasing pressure beyond the region of maximum registration my decrease the leak path opening. The embodiment of FIGS. 2A-2D may be configured so that the pressure of maximum registration is near the reasonable peak of physiological expiratory pressure, and therefore the variable opening leak path effectively only increases the leak path opening with increases in expiratory pressure over a physiological range. Thus, in general, the leak path opening during exhalation may be variable based on the pressure difference during exhalation. As the pressure difference increases the first and second element may separate or slide more (up to a point) and as the pressure difference decreases the first and second element may return to their initial, neutral, position, as shown in FIG. 3C.

In some variations, as pressure increases during exhalation, the leak path opens more (up to a point), increasing flow through the leak path during exhalation. This increased flow may decrease the resistance to exhalation at these pressures.

Another variation of a variable opening leak path is shown in FIGS. 4 and 5A-5B. This variation is formed from a single membrane that is configured (e.g., by cutting) to include a flow regulator that may be controllably deformed or expanded with increasing pressure across the nasal device, to increase the leak path opening as the pressure across the flow regulator increases. FIG. 4 shows the transition between a closed configuration (on the left) of the spiral-cut variable opening leak path at low expiratory pressure, and an opened configuration (on the right) at higher expiratory pressure. The level of the pressure across the device, and particularly across this variable opening leak path, may determine how much or how little the leak path is opened or closed.

Although FIG. 4 (and FIGS. 5A-5B) illustrate a variation with a spiral cut, variable opening leak paths may be formed in other patterns (e.g., non-spiral) patterns as well. For example, a variable opening leak path may include a plurality of cuts that form a pop-out region that can be displaced from the plane of the membrane into which they are cut. In some variations the cut pattern includes cuts that radiate inwards (along straight lines or curves) to form a pattern around a central (uncut) region that may billow outwards during exhalation. The central region may be any appropriate shape (e.g., round, oval, square, triangular, etc.). The lines forming the cuts may extend in radial and/or rotational pattern, relative to a reference center region. The spiral patterns described herein indicate just one variation of this.

In the example of FIG. 4, the flow regulator forming the leak path has a spiral design, formed by 12 curving cuts arranged in a spiral pattern. By comparison, the static leak pathways described previously were holes that were round or elliptical in shape. These static leak pathways had a relatively fixed shape, and do not substantially change their opening size as the pressure differential across them changes (e.g., increases during exhalation). For a static leak path in such a “hole resistor,” the flow can be approximated mathematically from the pressure: the pressure across the leak path is approximately proportional to the square of the flow through the leak path. Thus, the amount of pressure across the device, and the flow through the leak path during exhalation may depend on the size of the static leak path opening. A variable opening leak path may dynamically change this relationship between flow and pressure. As the area of the leak path increases at high pressure, more air may flow through the leak path. The variable opening leak paths described herein may be configured so that the opening of the leak path is larger at higher pressure (e.g., over the normal breathing range) resulting in a potential increase in flow (leak) through the leak path at high pressure. This may lower the resistance to exhalation at higher pressures such as may be present at the start of exhalation. This may make the devices more comfortable to wear, while still keeping the resistance to exhalation sufficiently higher than the resistance to exhalation to have therapeutic effects. This may also make the devices more effective, by enabling higher resistances at low flow rates to be employed while retaining satisfactory comfort.

FIGS. 5A and 5B, show an adhesive nasal device (configured as a “whole nose” nasal device, that may communicate with both nostrils) that includes an airflow resistor having eight flap valves, and a centrally located variable opening leak path configured as a spiral cut region just described in FIG. 4. In this variation the leak path opens more as pressure increases, allowing more flow with increasing pressure (as compared to a constant area orifice or “hole resistor). The spiral-cut flow regulator expands during exhalation to further open the leak pathway. As it opens, the central circular region may twist and extend out of the plane of the membrane forming the variable opening leak path. In this example, as in many of the other examples described herein, the same membrane may form the airflow resistor (flap valves) as the leak path (the variable opening leak path).

In FIGS. 5A and 5B, the mathematical relationship between flow and pressure may be different than that for a static hole resistor leak path over at least some range of pressures. For example, a variable opening leak path such as the one shown in FIGS. 4 and 5A-5B may have a pressure and flow relationship that is expressed as less than a second order relationship. For example, fitting a curve to the pressure and flow relationship may result in a curve fit in which the pressure may vary with the flow as a less than second order relationship over at least some range of pressures. For example pressure may be a function of an n^{th} power of flow, when the n^{th} power is 1.1, 1.2, 1.3, 1.4, 1.5, 1.6, 1.7, 1.8, 1.9, etc., but may be less than 2 over at least some range of pressures. In some variations the relationship approaches the linear. This may mean that for high pressure differentials during exhalation, more flow would pass through the variable opening leak path than a fixed opening leak path (e.g., a “hole resistor” leak path). The flow may still be limited, e.g., by the maximal opening of the variable opening leak path. In general, however, the resistance during exhalation is within the therapeutic range over at least a portion of the expiratory portion of the respiratory cycle, or the entire expiratory portion of the respiratory cycle, or during just the later portion of the expiratory portion of the respiratory cycle. Most importantly, the modulation of the flow through the leak path during exhalation is achieved passively using the variable opening leak paths described herein, without the addition of airflow from a gas source. For example, the resistance to exhalation through the entire device during exhalation may be between about 0.001 and 0.25 cm of H₂0/(ml/sec) measured at a flow rate of 100 ml/sec.

As mentioned, FIG. 5A illustrates one variation of a nasal device having a flow regulator that is cut into a spiral-shaped variable opening leak path similar to that shown in FIG. 4. In the neutral position, e.g., when no substantial pressure differential is present across the device, as shown in FIG. 5A, the leak path has a small opening and therefore a relatively small leak path. During exhalation, when the flap valves are typically closed, e.g., held closed against a flap valve limiter layer that is not visible in FIG. 5A or 5B, the spiral-shaped flow regulator pushed outward from the plane of the airflow resistor (e.g., out of the page in FIG. 5B). The expiratory pressure may apply force against the face of the central region 503, which may act like a sail to pull away from the plane of the airflow resistor and twisting along the cut lines 505, thereby increasing the leak openings. This variation of a passive nasal device may also include a peripheral adhesive holdfast 528, as mentioned above.

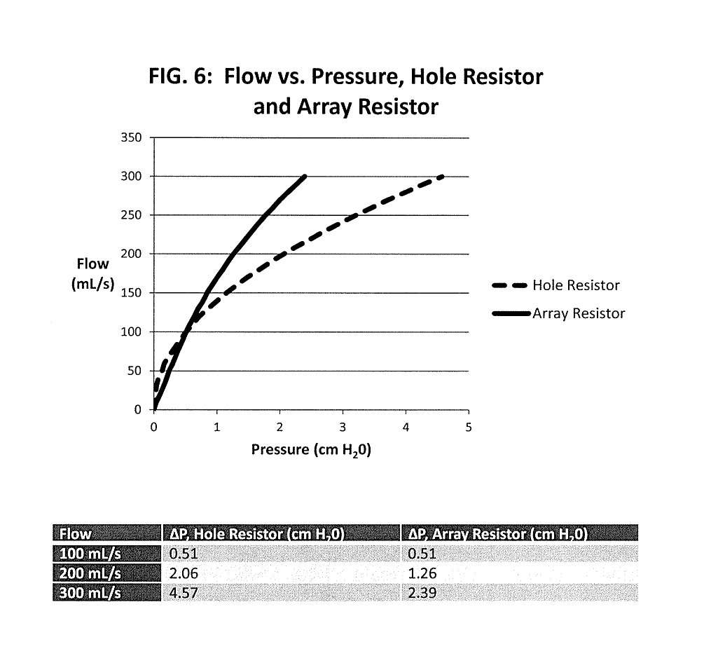

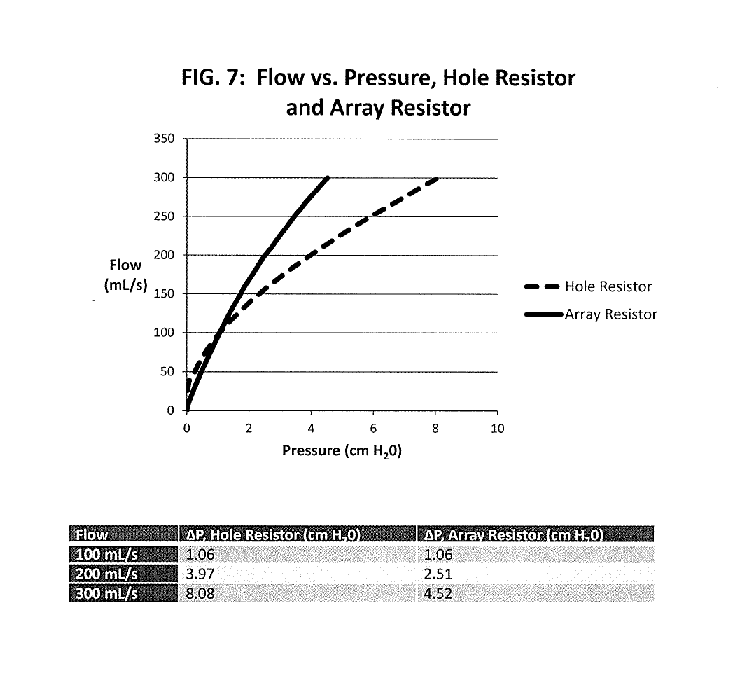

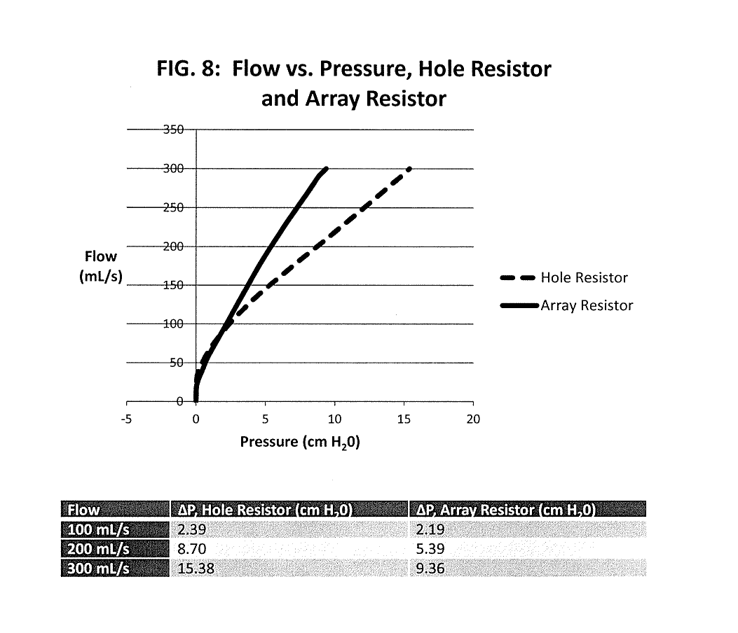

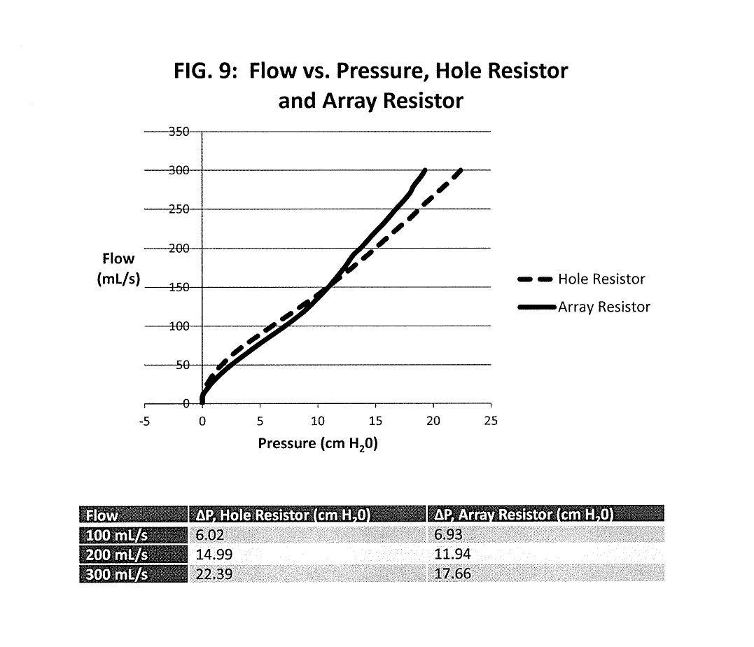

FIGS. 6-9 illustrate comparisons in the flow-pressure profiles for different variations of variable leak pathway devices (referred to as “array resistors”) having flow regulators compared to similar airflow resistors having static leak pathways (holes). All of these figures show a similar trend. For example, FIGS. 6-9 show comparisons between two devices, a static, fixed-size open leak path or hole resistor, and an variable opening leak path such as the one shown in FIG. 4, referred to as an array resistor. In FIG. 6, both devices are configured to have a flow of approximately 100 mL/s at a pressure of approximately 0.5 cm H₂O (thus the same resistance to exhalation at these parameters). In this example, the variable opening leak path device has a lower flow at pressures below 0.5 cm H₂O, and a higher flow at pressures above 0.5 cm H₂O compared to the static or hole resistor. Similarly, in FIG. 7 both devices are configured to have a flow of 100 mL/s at a pressure of approximately 1 cm H₂O. In this example, the variable opening leak path device has a lower flow at pressures below 1 cm H₂O, and a higher flow at pressures above 1 cm H₂O compared to the static or hole resistor. In FIG. 8, both devices have a flow of about 100 mL/s at pressure of about 2-3 cm H₂O, and the variable opening leak path has a similar, though slightly lower flow at pressures below 2-3 cm H₂O. In FIG. 9, both devices have a flow of about 100 mL/s at a pressure of between about 6-8 cm H₂O. In this example, there is a slight decrease in flow at pressures less than about 6-8 cm H₂O, and a slight increase in flow at pressures greater than about 6-8 cm H₂O.

In FIGS. 6-9, the devices include flow regulators cut into the spiral shape similar to those shown in FIGS. 4 and 5A-5B. Different flow regulators may have more or fewer cuts (curves or arcs) forming the spiral. In the variable opening leak paths of FIGS. 6-9, spirals having between about 3 and 12 arcs were used. The diameter of the inner circle (“sail” region 505 in FIG. 5B) varied between about 0.020″ and 0.15″. The outer diameter of the spiral region (at the ends of the cuts forming the arcs of the spiral) was between about 0.075″ and 0.3″. The angular offset from inner end of arcs to outer end of arcs was between about 15 degrees and 180 degrees, and the radius of curvature of arcs was ≧0.020″. These parameters were modified to set the resistance to exhalation though the leak path as indicated for each graph. By comparison, the typical hole diameter for the static leak pathways was between about 0.100 and 0.200″.

Part II: Nasal Devices with Deployable Insertion Guides

As mentioned above, some variations of the nasal devices described herein including a passive airflow resistor having one or more flap valves that open during inhalation and close during exhalation, therefore providing a greater resistance to exhalation than inhalation. Reliable operation of these devices may be assured by preventing blockage or interference of the flap valves during operation (e.g., during inhalation). However, in some variations, and particularly the relatively flat (e.g., layered) adhesive nasal devices, the devices may be worn directly against the outer region of the nose. Such devices may cover and/or partially insert into the subject's nostrils. The flap valves forming the airflow resistor may open during inhalation towards the subject's nostrils and must close during exhalation.

For example, a nasal device may include an insertion guide member that is configured to deploy from a collapsed configuration to an expended configuration that can be used to help guide the device into one or both nostrils. In some variations, the insertion guide member may also protect the airflow resistor (e.g., flap valves) by preventing material from contacting the airflow resistor and interfering with its opening and closing, and/or occluding a leak pathway. The insertion guide member may have a collapsed configuration that is planar and/or parallel to the airflow resistor and/or holdfast portions of the device. The insertion guide may be deployed so that prior to being inserted into the nose it extends out from the nasal device to form a guide region that can be used to guide insertion into the patient's nostril(s). Converting from a collapsed position to a deployed extended position may allow the device to be packaged more readily and at higher density. The nasal device may be stored before use (and/or packaged) on a card or backing, and the backing may be marked, scored, or otherwise indicate how to fold the card and/or device to deploy the insertion guide member(s). The insertion guides described herein may also be referred to as deployable alignment guides or, for convenience, alignment guides.

Thus, an insertion guide/alignment guide may be included to prevent interference between the airflow resistor, including the flaps of a flap valve, and the patient, e.g., the nares, nose hairs, septum, etc. The insertion guide may keep the airflow resistor aligned within the nostril(s) and also away from such interfering structures. In some variations the insertion guide also forms a protective region which may help block interfering structures from inhibiting the opening or closing of the airflow resistor.

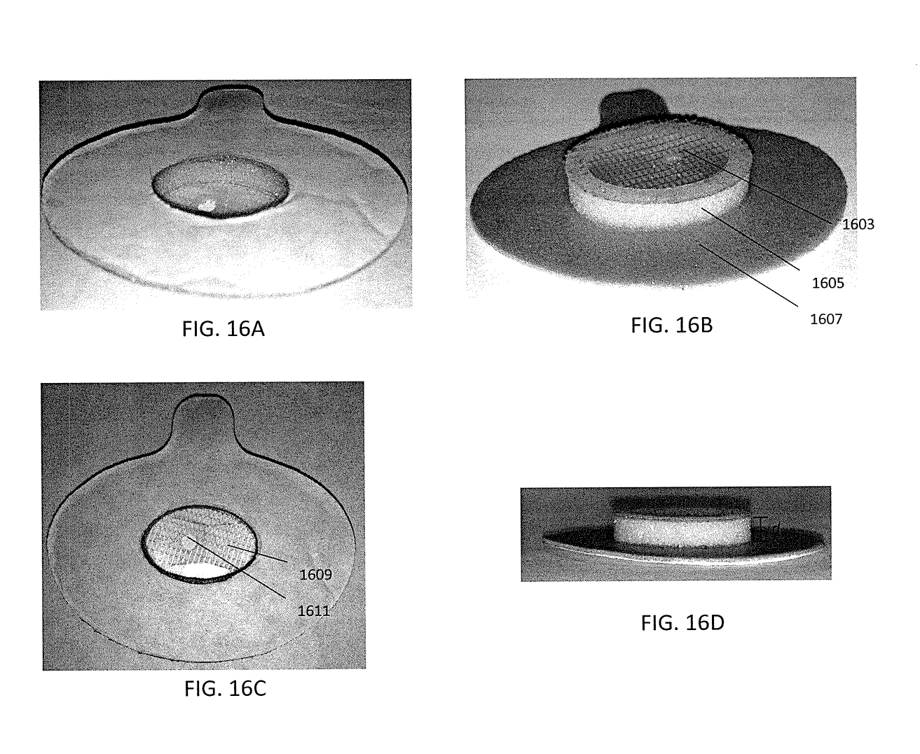

Although the deployable insertion guide members described are one solution that may prevent or help prevent interference of the airflow resistor by centering the devices in the nostrils and or blocking interference directly, FIGS. 16A-E, describe a second approach, in which the airflow resistor is held in a position that is away from the nostrils in a pop-out region (extension member) thereby allowing clearance for the flap valves. Either (or a combination) of these approaches may be used.

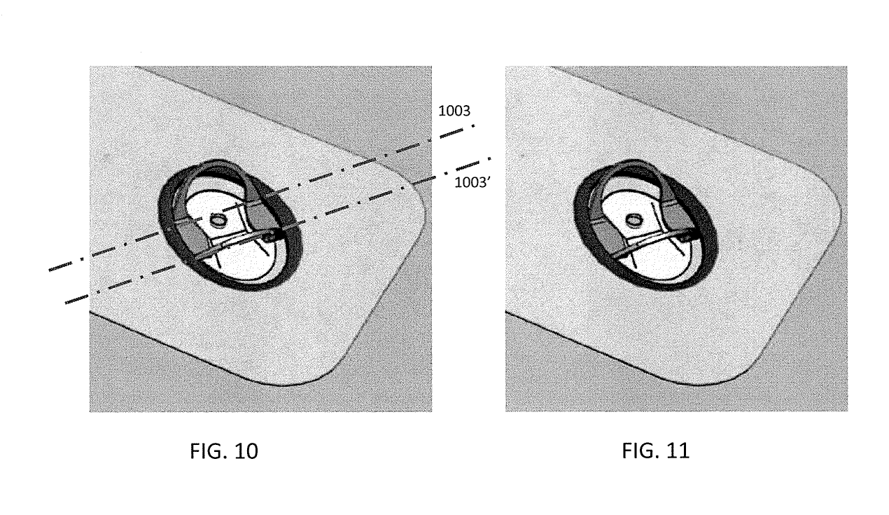

Referring now to FIGS. 10-13 and 14B-15E, in some variations, the insertion guide is configured as a ‘kickstand’ structure, or extendable frame, that can be extended from the nasal device, e.g., by the user or person applying the device, from a collapsed configuration into an expanded configuration. The arcs formed by bending the expandable frame out from the flat layer of the nasal device may be used to both align the device within the nostril(s) and/or to protect the flap valve from interference.

FIGS. 10-15E illustrate variations of this kickstand insertion guide element. For example, in FIGS. 10 and 11 the two-ring aligner/protection element may be extend by a user such as the patient, who may fold the nasal device along the two lines 1003, 1003′, to bend the insertion guide member away from the plane of the flap valves. The material forming the insertion guide member may be relatively deformable so that folding it in this manner may allow it to keep its shape after the rest of the nasal device returns to the relatively flat configuration, as shown in FIG. 11. The two rings forming the insertion guide may help with the application of the device into the nostril in the correct orientation, and/or block out interfering structures from within the patient's nose.

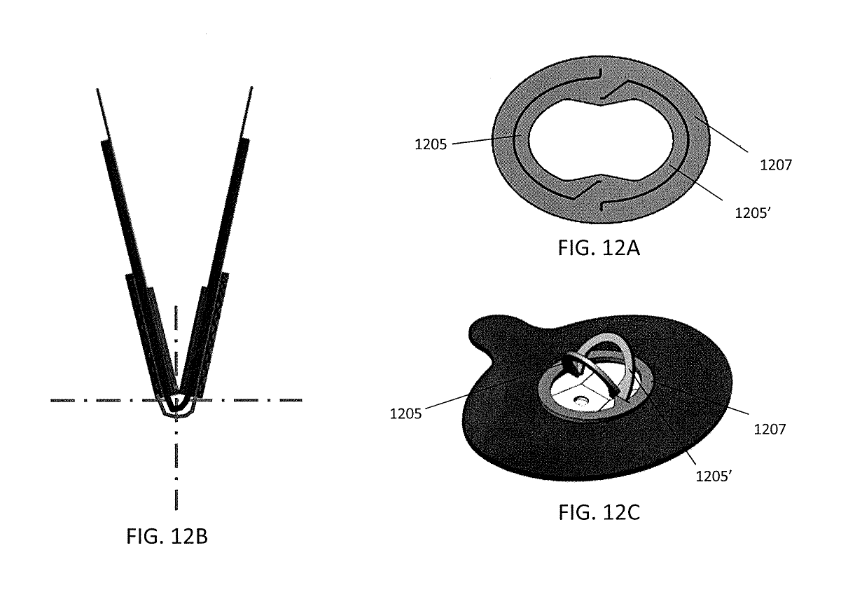

Another variation of a nasal device having a deployable insertion guide is shown in FIGS. 12A-12C. This example has an alignment guide (FIG. 12A) formed to have a pair of rings where the inner ring has two arched regions 1205, 1205′ that may be deployed from the plane of the flap valves by folding the device (prior to application) as shown in FIG. 12B. The outer ring 1207 may remain secured to the rest of the device, such as the holdfast region, as shown. The cut-out form of the deployable insertion guide or region shown in FIG. 12A may be included into the nasal device. FIG. 12C shows the rings of the insertion guide deployed form a nasal device.

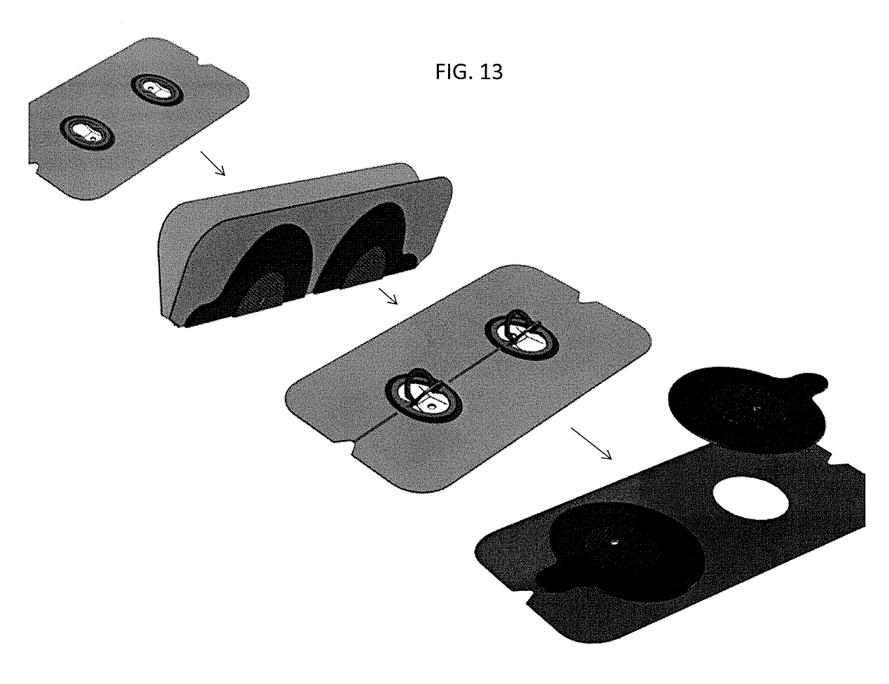

FIG. 13 also illustrates a pair of nasal devices (one for each nostril, in this variation) on a backing card support that is configured to be folded prior to application of the nasal devices to the patient's nose, in order to deploy the aligner/protective elements of the nasal devices. After deploying, the devices may be peeled off and applied to each of the subject's nostrils, using the deployed rings as guides.

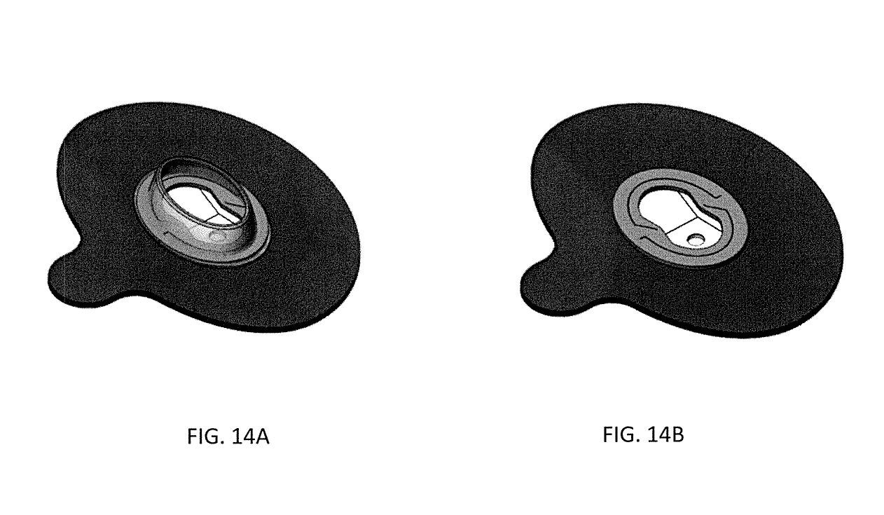

FIG. 14A compares a nasal device having a “cone” type alignment guide with one variation of a foldable/deployable insertion guide such as the one shown in FIG. 12A-12C. The deployable aligner/protective element of FIG. 14B has a similar footprint on the nasal device as the cone aligner shown in FIG. 14A.

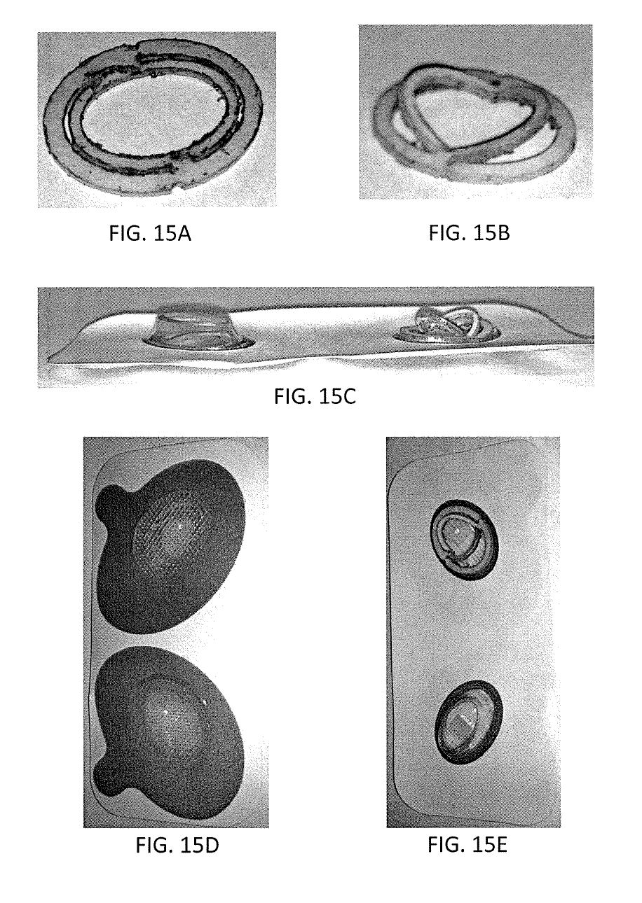

FIG. 15A shows another example of a deployable insertion guide member in an un-deployed state. For comparison, FIG. 15B shows the same structure in the deployed configuration. In this example, the inner arcs of the kickstand insertion guide are deployed by pulling the inner arcs out of the plane of the structure (which is parallel with the plane of the flap valves of the airflow resistor). FIG. 15C shows a side perspective view of the comparison between the deployable insertion guide member (right) and a cone aligner (left). FIG. 15D also shows a front view (looking as though towards a patient wearing them) of these two examples of nasal devices shown in FIG. 15C. FIG. 15E shows a back view of the same two devices shown in FIG. 15C.

Other variations of deployable insertion guide members may include a single arc, or members that are not arced. In some variations the deployable insertion guide member cross over the airflow resistor, which may protect the airflow resistor, as show in FIGS. 15A-15E, however in some variations the deployable insertion guide member does not cross over the airflow resistor, but extends adjacent (or partially over) it.

Part III: Nasal Devices with Extension Members

In some variations a passive nasal device may include an extension member that holds the airflow resistor near, but apart from the openings of the nostril. This separation between the airflow resistor and the nostrils may be minimized so that the device is small, lightweight, unobtrusive and comfortably worn, but is large enough that any structures associated with the nose (hairs, mucus, the sides of the nostrils, etc.) will not interfere with the opening and closing of the airflow resistor. These variations may also allow a single-sized nasal device to be used with a wider variety of patient sizes, despite the wide variety of nose, nostril, and nostril-opening sizes possible in the average patient population.

For example, FIGS. 16A-16D illustrate another variation of a nasal device having an airflow resistor in which the flap valves have been offset away from the patient's body in a pop-out region, to prevent them from getting interference from the patient's nose or other structures. The pop-out region is formed by an extension member 1605 projecting from the outward-facing side of the passive (and in this variation layered) nasal device. This variation of an extension member is shown as an oval ring having a central opening. The top surface of the oval is attached to the airflow resistor 1603, which in this example is a flap valve having two flaps that open during inhalation upwards in FIG. 16A, and downward in FIG. 16B) and close against the flap valve limiter layer visible in FIG. 16B. This embodiment may be referred to as having an offset airflow resistor.

Nasal device in which the extension member holds the airflow resistor at least some minimum distance (e.g., 1/16^{th} of an inch) from the subject's nose, such as those shown in FIGS. 16A-16D may eliminate or reduce the need for an alignment feature, since the offset allows the airflow resistor to open even if the nostril openings and the airflow resistor are not exactly aligned. Thus, nasal devices having such an offset airflow resistor may be more forgiving of placement, having a higher tolerance for placement variability. Although these variations are described with respect to flap valves, other passive airflow resistor valves may also benefit from these designs.

In FIGS. 16A-16D, the extension member (offset) is formed by a ring of foam or other material. In some variations the material is a lightweight form. The extension member may be solid or porous (e.g., providing leak path(s)). As mentioned, the pop-out configuration of the extension member may keep the nose structures from interfering with a flap valve of an airflow resistor. In variations having a flap valve, any appropriate flap design may be used.

The extension member may be configured to hold the airflow resistor any appropriate distance from the subject's nose (e.g., nostril openings). The thickness of the extension member may set this distance. For example, the minimum pop-out thickness, e.g., the minimum distance from the plane of the airflow resistor to the plane of the holdfast, may be greater than about 1.5 mm. As mentioned, this minimum distance may be related to the size of the airflow resistor in the open state; for example, the minimum distance may be approximately greater than the distance that the open airflow resistor valve (e.g., flap valve) projects from the plane of the airflow resistor in the closed state. Thus, larger size valves may have a greater minimum distance (e.g., 2 mm, 2.5 mm, etc.). In some variations, the extension member has an upper limit in the thickness or distance between the region to which the airflow resistor is attached (e.g., the top region) and the region connected or forming a part of the holdfast securing the device to the patient (e.g., the bottom, nose-contacting region). This maximum distance may be, for example, less than about 50 mm, less than about 30 mm, less than about 25 mm, less than about 20 mm, etc. For example, the extension member may be configured to secure the airflow resistor from between about 1.5 to about 25 mm from the subject's nose.

FIG. 16A shows, a bottom perspective view of the device with the pop-out region formed by the extension membrane 1605 extending from the adhesive holdfast 1607. The back side of the adhesive holdfast (including a backing paper protecting the adhesive) is shown, and a cavity formed by the extension member is visible. The opening into this cavity or chamber is oval, though other shapes may be used. In FIG. 16A, the airflow resistor is a flap valve including a fixed open holdfast through which air flows. The flap valve is attached to the opposite side of the extension member 1605 from the adhesive holdfast, and the formed cavity provides a space into which the flaps may open and close without interference.

FIG. 16B shows a perspective view of the opposite side of the nasal device, and is labeled to indicate the airflow resistor 1603 (including a flap valve layer and a valve limiting layer) attached to a first end of the extension member 1605 and the adhesive holdfast 1607 attached to the opposite side of the airflow resistor.

FIG. 16C shows a back (or bottom) view of the airflow resistor, similar to the view shown in FIG. 16A. As mentioned, the flap valve layer 1609, leak path 1611 (shown as a central static opening, though the variable or dynamic leak paths described above may be used), and flap valve limiter layer are all positioned on a first end of the pop-out extension member (configured as an oval ring). The holdfast region (adhesive holdfast) is shown on the other end.

FIG. 16D is a side perspective view of the nasal device, showing the airflow resistor layer is separated from the holdfast by a distance, d. As mentioned, this distance, which may be referred to as the thickness of the extension member, may be greater than about 1/16^{th} of an inch or about 1.5 mm. In some variation, this thickness is less than about 35 mm (e.g., less than about 30 mm, less than about 25 mm, less than about 20 mm, etc.). Although the examples shown in FIGS. 16A-16D are single-nose devices for application over and/or against a single nostril, whole-nose devices that may interface with both of a subject's nostrils may be used.

In general, any of the devices described herein may be worn, operated or used by a patient. A patient may also be referred to herein as a subject or user, and may include any appropriate patient particularly humans. The devices may also be used by non-human (e.g., veterinary) patients.

While the devices (and methods for using them) have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

1-34. (canceled)

35. A passive-resistance nasal device, the device comprising:
an airflow resistor configured to provide a resistance to exhalation that is greater than the resistance to inhalation; and
a variable opening leak path through the device configured so that the size of the leak path opening increases as expiratory pressure increases and decreases as expiratory pressure decreases.

36. The device of claim 35, wherein the nasal device is configured to have a resistance to exhalation that is between about 0.002 and about 0.25 cm H₂O/(mL/sec) when measured at 100 mL/sec.

37. The device of claim 35, wherein the device is an adhesive nasal device comprising an adhesive holdfast configured to secure the airflow resistor in communication with one or both nostrils.

38. The device of claim 35, wherein the variable opening leak path comprises a flexible membrane.

39. The device of claim 35, wherein the variable opening leak path comprises a pair of membranes, wherein at least one of the membranes is configured to slide relative to the other as expiratory pressure increases and decreases.

40. The device of claim 35, further wherein the variable opening comprises a membrane having a spiral of curved cuts.

41. The device of claim 35 wherein the airflow resistor comprises at least one flap valve.

42. The device of claim 35 comprising a holdfast region configured to secure the device in communication with both nostrils.

43. A passive resistance nasal device for use while sleeping, the device comprising:
an airflow resistor configured to provide a resistance to exhalation that is greater than the resistance to inhalation; and
a variable opening leak path comprising a membrane having a plurality of cuts forming leak path openings through the membrane arranged in an approximately circular array and configured so that the size of the leak path openings increase as expiratory pressure increases and decreases as expiratory pressure decreases.

44. The device of claim 43, wherein the nasal device is configured to have a resistance to exhalation that is between about 0.002 and about 0.25 cm H₂O/(mL/sec) when measured at 100 mL/sec.

45. The device of claim 43, wherein the device is an adhesive nasal device comprising an adhesive holdfast configured to secure the airflow resistor in communication with one or both nostrils.

46. The device of claim 43, wherein the membrane of the variable opening leak path comprises a flexible membrane.

47. The device of claim 43, wherein the airflow resistor comprises at least one flap valve.

48. The device of claim 43, comprising a holdfast region configured to secure the device in communication with both nostrils.

49. A method of treating a sleeping patient, the method comprising:
applying a passive nasal device in communication with one or both of the patient's nostrils without covering the patient's mouth;
inhibiting exhalation through the patient's nose more than inhalation through the nose; and
changing the size of a leak path opening through the nasal device during exhalation based on the pressure applied across the nasal device during exhalation.

50. The method of claim 49, wherein applying comprises adhesively applying the nasal device.

51. The method of claim 49, wherein inhibiting exhalation comprises closing a flap valve during exhalation.

52. The method of claim 49, wherein changing the size of the leak path comprises expanding an array of leak path openings configured so that the size of the leak path openings increase as expiratory pressure increases and decreases as expiratory pressure decreases.

53. The method of claim 49, wherein changing the size of the leak path comprises expanding or collapsing an array of spiral of curved cuts so that the size of the leak path openings increase as expiratory pressure increases and decreases as expiratory pressure decreases.

54. The method of claim 49, wherein changing the size of the leak path comprises sliding a first membrane relative to second membrane as expiratory pressure increases to open the leak path.
